(19) United States
(12) Patent Application Publication
(10) Pub. No.: US 2006/0015016 A1
(43) Pub. Date: Jan. 19, 2006

(54) Caloric balance weight control system and methods of making and using same

(76) Inventor: William E. Thornton, Boerne, TX (US)

Correspondence Address: ROBERT W STROZIER, P.L.L.C, PO BOX 429, BELLAIRE, TX, 77402-0429, US

(21) Appl. No.: 11/159,421

(22) Filed: Jun. 22, 2005

Related U.S. Application Data
(60) Provisional application No. 60/581,983, filed on Jun. 22, 2004.

(51) Int. Cl. A61B 5/00; G06Q 10/00; G01G 23/18

(52) U.S. Cl. 600/300; 177/25.19; 128/921; 705/2

(57) ABSTRACT

A new system and methodology is disclosed for developing interactive weight control and maintenance programs, where an weight control program is developed during a visit to a treating physician or expert based on medical data, medical historical data, and patient profile data. The data and program is then stored in a host computer and a relevant portions are transferred to a weight measuring and feedback unit including a scale and a processing unit having audio and/or visual and/or tactile output devices for patient feedback, which is taken to the patient's home. The patient then weighs himself/herself on a periodic basis and the apparatus issues recommendations or other messages for improved compliance.

### RELATED APPLICATIONS

This application claims priority to U.S. Provisional Patent Application Ser. No. 60/581983, filed 22 Jun. 2004.

### BACKGROUND OF THE INVENTION

1. Field of the Invention

The present invention relates to a system for implementing individual weight control programs.

More particularly, the present invention relates to a system for implementing an individual weight control program including processes and means for daily monitoring of a patient's compliance with the program and assisting patient adherence to the program and to method for implementing the system.

2. Description of the Related Art

There is now a large and growing mass of scientific and medical documentation of the serious conditions and diseases caused by excess weight and an equally well documented, increasing pandemic of excess weight in many countries, especially the United States of America. The medical and economic impacts of this pandemic are large and increasing but the full impact will be felt in say 20 years. A staggering increase in patient morbidity and medical costs.

None of the innumerable existing weight control programs have not made a significant impact on the problem, in spite of the clamor and billions of dollars spent. There are several reasons for this failure including; weight control is a complexm, multi-factorial problem, but most programs place emphasis on only one or two factors. Most, if not all, monitoring systems are inadequate and most programs attempt rapid, large and unphysiologic changes. Few programs have any follow up or monitoring component to sustain any losses attained. Most programs are expert or supervisors, rather than patient oriented and require large amounts of time and/or significant changes in patient life style.

Some examples includes special or proprietary diets such as the low carbohydrate, high protein low “carb” Atkins diet, calorie counting and weekly weighing done randomly and in clothes by Weight Watchers, “Slim” and many other diet preparations, a variety of exercise programs, programs based on basal metabolism, programs based on drugs, etc. While most programs can transiently produce more or less weight reduction and a few subjects usually experience long term reductions, the great majority, an estimated 95%, of subjects relapse even with the most advanced medical programs.

Basal human weight is normally determined by the energy difference between diet and activity for the body has internal stores of energy in the form of fat that are increased or decreased according to balance between diet and activity. This usually produces corresponding changes in body weight. It is well established that excessive losses or gains of fat are harmful.

In the past and in much of the world today, the energy cost of obtaining food is high and prevents excess gains in fat. Today, in the U.S. and some other countries, technology and subsidies have reduced the cost of food such that an excess is now freely available. In addition, purveyors of food have mounted preparation and sales techniques that have effectively increased food intake far beyond that required for much of our population. Technology has also reduced other demands on human activity and energy cost. The net effect is that there is now a large and growing imbalance between energy intake with an excess of body fat and fat induced disease in an increasing percentage of the population, in spite of increasing efforts and money spent to control the problem.

The problem of weight control is now one of self-control involving many factors including: (1) individual personality and psychology; (2) individual dietary habits; (3) individual physical activity habits; (4) individual knowledge of weight control; (5) individual means of detecting energy imbalance; (6) individual psychological support for weight change; (7) practical and feasible system for implementing change; (8) practical and feasible system for maintaining change; (9) differences in individual utilization of energy; (10) genetic differences, especially in satiety and hunger; (11) external marketing pressures; and (12) social mores.

Thus, there is an urgent need in the art for a system that considers all aspects of the problem: diet, activity, patient motivation and variation, patient time, and which correctly utilizes physiological and other knowledge, plus state of the art technology.

### SUMMARY OF THE INVENTION

The present invention provides a system for implementing an individual weight control program including a home unit for daily monitoring of a patient's compliance with an individualized weight control program and for assisting patient compliance with the program.

The present invention also provides a method for implementing an individual weight control program including the steps of consulting with a physician, healthcare provider or other expert to ascertain goals and objectives, gathering data in a data gathering session, evaluating the gathered data, and generating an individualized weight control program. Next, the program and associated personalized data are entered into a digital processing unit (DPU) associated with an assistant/coaching/monitoring apparatus, where the monitoring apparatus includes a stable, sensitive computer augmented scale in data communication with the DPU. Once the program has been loaded into the DPU, the monitoring unit is installed in the patient's domicile. After installation of the monitoring unit in the patient's domicile, the patient weighs himself/herself daily, preferably within a specific time window, in the nude and after voiding. Next, the program computes weight difference data and daily trend data and compares the daily weight and daily weight trend data to the program target weight profile and plotting the target weight control trend line, the daily weight data and the daily trend weight data, preferably from a mean change in weight based on several days of weight data. Based on the direction and magnitude of the difference between the target profile and the daily profile, the monitoring unit issues a message or a plurality of messages, visually and/or verbally, to the patient tailored to improve compliance with the program. Generally, the message(s) is designed to suggest changes in routine, diet, exercise, and/or lifestyle so that the patient can meet the target profile. Additionally, the message(s) is designed to encourage and aid the patient in attaining the target profile and final target weight. The unit also stores the daily and processed weight data in a file. Periodically, the stored data is transmitted to the program director (physician or expert) for review. During the review of stored data, the program director can modify the program, uploading any such modifications to the domicile based monitoring unit and, if necessary, request a follow up consultation with the patient to correct any program or behavioral deficiencies. Once a desired weight goal has been achieved, the program becomes a maintenance program with no desired change in weight, i. e., the weight control trend line is a horizontal line at the target weight and the daily weight data and computed weight values are plotted relative to the target weight trend line. The maintenance program still requires daily weighs and still generates commentary, but it only transmits weight data to the healthcare provider if a significant weight change trend occurs. Minor fluctuations in weight are not considered significant weight change trends.

While the system and method can be used to gain, loose or maintain weight, the system and method will more often be used in a program to loose weight rather than gain or maintain weight; however, the same basic process is employed.

The present invention provides a method for designing, monitoring and assisting a weight control program including the steps of an initial physician or expert consultation, examination and data gathering session, where a patient is examined by a physician, healthcare provider or expert to obtain initial medical data and patient profile data. The patient profile includes both psychological and physiological data as well as medical data and the profile is used by the program to determine a best set of messages that will be generated during the weight control program's non-maintenance phase to suggest changes in diet, exercise and lifestyle based on the profile to achieve the desired end result and to provide encouragement to aid the patient in achieving the desired end result. After data acquisition, the physician or expert and patient develop a weight loss or gain or maintenance program including daily modulation of the individuals usual diet and activity to achieve a desired or target weight loss trend. An amount of loss or gain over a reasonable time period is selected and the resulting desired or target rate of loss or gain is stored in memory of the monitor, with patient characteristics including some psychological, diet habits, exercise habits and attributes of physical conditions of the patient.

Next, the patient then weighs daily under standard conditions, fixed time of day and no clothing, using the monitoring unit which then calculates a sliding weight trend, displayed graphically as well as the desired trend and daily weight, which is compared to the desired trend. Depending on the magnitude and direction of the trend and dietary profile, activity profile, patient's recorded personality profile, a synthesized voice message, encouraging, congratulatory, chiding, etc. is audibly delivered to the patient. In addition suggested modulation of the usual patient diet and activity profiles, based on stored data and desired change, is also delivered audibly. At a previously scheduled time, the patient will transfer data from the monitor to the program expert where it will be received, and if necessary, the program modified and the modification uploaded to the monitor. If and when the desired goal is reached, a stable weight monitoring program is continued indefinitely. The monitoring program after a stable weight is attained is simple. The entire time required is approximately 1 minute per day and becomes a part of the patient's normal daily activities.

Should the modulation of normal activities be inadequate more extensive monitoring of food and activities may be done as well by ambulatory monitors such as described in U.S. Pat. No. 5,263,491, incorporated herein by reference, and more rigorous control employed. Also the home monitor can be programmed with more rigorously defined and prescribed diets and exercise programs. Although not described at this time, the monitor may be interactive and accept input from the patient or from the patient's home monitor. Also the system may be used in either a family or in an institutional setting, because the system can accept multiple files and discriminate between patient weight differences, or if necessary it can be provided with coded inputs for each patient.

The present invention also provides a method for designing, monitoring and assisting a weight control program including the steps of an initial physician or expert consultation, examination and data gathering session, where a patient is examined by a physician or expert to obtain initial medical data and patient profile data. After data acquisition, the physician or expert and patient develop a weight loss program including changes to diet and changes to exercise to achieve a desired or target weight loss trend. The data and trend are then entered into a scale apparatus including a computer which is taken home with the patient and installed in the patient's domicile. The patient then weighs himself/herself on the apparatus periodically, preferably daily. The apparatus then compares the measured periodic weight against a target weight value given by the target trend for the same period. Based on the comparison, the apparatus issues a message to the patient to assist the patient in achieving the target trend and final target weight goal. The apparatus also stores the periodic weight measurements in a data file. The stored data is then periodically reported and/or transmitted to the treating physician or expert. At least one return physician or expert view is scheduled during the term of the program to access the success of the program and to determine if the program needs to be modified. Any modifications are then fed into the apparatus.

The present invention provides a system for controlling a patient's weight comprising a weight loss program developed from patient medical data and patient profile data, an interactive weight measuring, monitoring and feedback apparatus including a computer having the program implemented therein, and periodic patient weighing to determine patient compliance with the program and to generate messages designed to facilitate patient compliance.

The present invention also provides a system for determining optimal weight loss programs including a database having programs, compliance data, patient medical data and patient profile data and a computer program acting on the database to generate patient categories and to optimize weight loss programs for each patient category.

### BRIEF DESCRIPTION OF THE DRAWINGS

The invention can be better understood with reference to the following detailed description together with the appended illustrative drawings in which like elements are numbered the same:

FIG. 1 depicts a preferred embodiment of a monitoring apparatus of this invention;

FIG. 2A depicts a preferred configuration of the monitoring apparatus of FIG. 1,

FIG. 2B depicts another preferred configuration of the monitoring apparatus of FIG. 1,

FIG. 3 depicts a conceptual flow chart illustrating a preferred entry and creation of an individually tailored weight control program of this invention;

FIG. 4 depicts a conceptual flow chart illustrating a preferred implementation of the weight control program of FIG. 3;

FIG. 5 depicts a conceptual flow chart illustrating a preferred review and modification of the weight control program of FIG. 3;

FIG. 6 depicts a conceptual flow chart illustrating a preferred method for generating and optimizing patient categories and weight control and maintenance programs for each category;

FIG. 7A-D depict plots of a model set of data showing actual periodic data, trend data and program target data, showing monitor installation and data display as weighings continue; and

FIG. 8 depicts a plot of a second model set of data showing actual periodic data, trend data and program target data.

### DETAILED DESCRIPTION OF THE INVENTION

The inventors have found that a method and system for improving a patient's compliance with a weight loss and/or gain program can be implemented using a combination of medical interaction and system interaction. The method and system requires a treating physician or expert to gather medical data and patient profile data in an initial physician or expert consultation. Most of the data is gathered by completion of standard questionnaires or forms and the gathered data is transferred to the program in a mixture of manual and automated data input. From the medical data and profile data and interaction between the patient and physician or expert, a weight loss/gain program is developed including a weight loss/gain target trend. The trend and profile data is then entered into an interactive weight measuring device designed to be taken home with the patient and installed in the patient's domicile. The apparatus includes a computer capable of receiving weight data from the weight measuring device, comparing the measured data to the trend data value for the same period and generating a message to the patient based on the comparison.

The present invention broadly relates to a method for designing, monitoring and assisting a weight control program including the steps of an initial physician or expert consultation, examination and data gathering session, where a patient is examined by a physician or expert to obtain initial medical data and patient profile data. After data acquisition, the physician or expert and patient develop a weight control program including changes to diet and changes to exercise to achieve a desired or target weight control trend. The data and trend are then entered into a weight measuring apparatus which is taken home with the patient and installed in the patient's domicile, where the apparatus includes a computer. The patient then weighs himself/herself on the apparatus periodically, preferably daily. The apparatus then compares the measured periodic weight against a target weight value given by the target trend for the same period. Based on the comparison, the apparatus issues a message to the patient to assist the patient in achieving the target trend and final target weight goal. The message can be an audio message, a visual message, an audiovisual method or a mixture or combination thereof. The apparatus also stores the periodic weight measurements in a data file. The stored data is then periodically reported and/or transmitted to the treating physician or expert. At least one return physician or expert view is scheduled during the term of the program to access the success of the program and to determine if the program needs to be modified. Any modifications are then fed into the apparatus. Normally, the only modification to the system would be to change the slope of the prescribed weight change to a horizontal line. The method can also include the step of modifying the program specifics to better tailor weight loss programs to particular patient categories based on the initial medical data, profile data and program response data. The resulting database can then be used to determine statistically significant weight loss control parameters and patient categorical parameters. Normally, the only modification would be to change the slope of he prescribed weight change line to a slope of zero.

The present invention broadly relates to a system for controlling a patient's weight comprising a weight control program developed from patient medical data and patient profile data, an interactive weight measuring, monitoring and feedback apparatus including a computer having the program implemented therein, and periodic patient weighing to determine patient compliance with the program and to generate messages designed to facilitate patient compliance.

The present invention also broadly relates to a system for determining optimal weight control and maintenance programs including a database storing weight control and maintenance programs, compliance data, patient medical data and patient profile data and a computer program acting on the database to generate and optimize and/or modify patient categories and corresponding weight control and maintenance programs.

Besides the professional system, the inventor has also found that two simplified versions of the system can be constructed, one is a semi-professional system and the other is a do-it-yourself system.

The do-it-yourself system is roughly equivalent in function to the professional system. The do-it-yourself system includes a battery operated scale with wire or wireless data link to a computer, where the wireless can be Y5, IR, RF, ultrasound or any other wireless communication protocol. The computer can be any digital processing unit having a processor, a memory, an optional mass storage device, communication hardware and software, and peripheries such as a display unit, a data entry unit such as a keyboard, touch screen display, microphone, speakers, or the like. The do-it-yourself system will generally not includes voice and personal background data components that are part of the professional system. The do-it-yourself system will generally include a plurality of selectors such as push buttons (PB) such as reset, weight goal, etc. and maybe capable of displaying multiple weight loss or gain target profiles. The reset button will permit the user to reprogram the system for a different weight goal. The do-it-yourself system includes a manual with generic food and exercise values designed to provide the user with suggestions for daily diet and exercise. The system also includes a memory stick having a capacity sufficient to store data and program updates. The data stored onto the memory stick can be transferred to a normal computer for data analysis and/or printing.

The semi-professional system includes all the components and features of the do-it-yourself system, but also includes a voice recommendation package based on weight, subject specific values and subject specific food/activity data, i.e., the semi-professional system generally does not include individual psychologic, food, activity data or the like. Again, the system includes a memory stick, but in the semi-professional system, the memory stick will be used to periodically transfer the weight data to a host computer for review and analysis of the weight data by a nutritionist. Again, the memory stick can also be used to transfer the weight data to a personal computer for analysis and/or printing.

The professional system or any of the other systems can also include a restart feature that allows the user or a health care professional to reset the original prescription and the original Rx line to the current mean weight value or mean value weight trends. This feature is especially useful in cases where the slope of the actual mean weight trend deviates widely from the originally Rx line. Any one of the systems can also feature an option for the display of a plurality of Rx lines having different weight trends to accommodate users the loss or gain weight at different rates. Although this option could be implemented on any of the systems, the option is preferably implemented on the do-it-yourself system. Another feature that the systems can have is that the Rx line can automatically transition from the Rx goal trend line to an Rx line having a zero slope when mean target weight is reached. The systems can also include other features such as highlighting actual mean weight line, when the actual mean weight line deviates from the Rx line by a preset or user set amount.

The present invention provides for a continuum of change from day to day weight measurement data. The present invention includes specificity based on detailed dietary and activity data as well as generic data (sex, age, etc.); thus, the feedback with the present system is timely and vocal plus graphics. The present invention includes a number of techniques to achieve a meaningful sensitivity to small changes in daily weight, after arising, voiding and in the nude. after void and bare. Current system such as a system from Soehnle et al and from Conair, Inc., are not capable of trend line analysis and monitoring and do not include the specificity of information and weed back offered by the present invention.

Caloric-Balance Approach

Gain or loss of weight is determined by a simple relation:
Change in weight=A×caloric intake [food]−B×caloric expenditure [total work]
A and B are individual patients' constants, determined by many factors including the patient's weight. The highly individual nature of these two constants is often lost sight of and the result is “one size fits all programs” which do not work.

Weight control begins and ends with control of intake of food and external work done, i.e., exercise or physical exertion. It is the control of intake verses expenditure that allows a patient to successfully complete a desired weight control program. Thus, these two factors are critical to achievement of a weight control program and need to be controlled, preferably on a regular basis and particularly, on a continuous basis. Although monitoring these two factors directly may be problematic, measuring the result according to the above equation is simply a matter of taking a meaningful weight measurement. The information obtained from monitoring a patient's weight and reporting the results of the monitored weight relative to a target value allows the patient to adjust behavior to better comply with the target trend and provides vital information and data to the treating physician or expert allowing the physician or expert to assess compliance and possibly modify the program based on the monitored data.

The present invention represents an effective negative feedback control system and methodology for weight control, i.e., weight gain or loss depending on the patient. In the privacy of the patient's domicile (e.g., bathroom or bedroom), results of periodic, preferably daily, changes in weight are compared to a desired or target change based on the patient's weight control program and a difference between the actual and desired value is computed. From these differences and based on the patient's individual profiles (diet, activity, psychological, etc.), the apparatus issues suggestions and/or issues messages to the patient on ways to correct the difference and/or issues messages of encouragement to aid the patient in successfully completing the program. Of course, the corrective action is entirely patient dependent. A highly motivated patient will act on suggested changes and correct the error, while other patients may take partial or no corrective action. A weight correction program will succeed or fail based on the patient's actions.

A number of features have been designed into the present invention to optimize patient compliance. One such feature is to treat the problem as a medical problem or condition from the beginning (ab initio) under a physician's or other expert's supervision and control. The program is further enhanced by fitting it to each individual. Another key features of the programs of this invention is having a physician's surrogate, the weighing apparatus, present at every weighing, permitting audio, visual and/or audiovisual feedback to the patient from the computer associated with the surrogate. The feedback is based on the patient's medical data, psychological, dietary and activity profile data, historical data, weight control program, and compliance data (periodic differences between actual weight and target weight). The messages can include: (1) lifelike verbal instructions and/or words of encouragement to the patient; (2) visual instructions and/or visual encouragements to the patient; and/or (3) audio visual instructions and/or words of encouragement to the patient. The apparatus can be fully interactive, partially interactive or non-interactive depending on the patient, the program, program compliance, the physician or expert, or the like. Preferably, during a normal weight control program, the apparatus is partially interactive. By fully interactive, the inventor means that the apparatus will not only issue messages, but will also respond to the patient based on certain spoken words, collections of words, or phrases giving additional messages. Moreover, fully interactive can optionally include direct access to a healthcare provider including, without limitation, the treating physician or expert, his/her stand in, his/her assistant or the like via phone or email. By partially interactive, the inventor means that the apparatus will issue messages, but will respond only to weight measurements, not to verbal questions or comments. By non-interactive, the inventor means that the apparatus does not issue messages or comments in response to verbal questions or comments from the patient.

The caloric balance approach is based on periodic comparison of a patient's actual mean weight trend and target weight trend, displayed as two graphs on the monitors display. By reviewing the difference between the actual mean weight trend and the prescribed trend, the patient's program compliance can be visualized by the patient and can be transmitted to the patient'health care provider for modification. The shortest practical sampling period for monitoring weight change is the diurnal cycle, i.e., daily weighings, the preferred period for the caloric balance approach of this invention.

A brief rationale for some of the core concepts of a preferred program of this invention follows. First, the patient makes an initial visit to a physician or expert, where an extensive survey is made of patient's relevant psychological characteristics, dietary and eating patterns or habits, activities and exercise patterns or habits, life style, etc., a few basic measurements such as estimated body fat, key girths, general fitness, etc. The patient is then optionally subjected to an examination to measure blood pressure, heart rhythm, blood parameter (cholesterol, blood sugar, etc.) and to assess pre-existing medical conditions. This data (medical and profile data) is then entered into a computer located at the physician's or expert's office, called the expert computer herein, and a weight control program is developed and stored. The program and subject specific data is then input into the weight measuring and feedback unit, which the patient takes home. Periodic weighings are used to determine patient compliance and to activate the feedback portion of the program. The feedback includes audio, visual or audiovisual messages to the patient to advise the patient on how to better comply with the program, i.e., suggested changes to exercise programs and eating habits, to encourage the patient to continue with the program and to encourage continued compliance with programs, and to illustrate differences between compliance and non-compliance. The interactive weighings continue until the program is completed successfully. After successful completion, the slope of the prescribed trend line is set to zero and periodic weighings are continued. The apparatus still collects and stores the weight data and transfers the data periodically to the host computer. This maintenance part of the program allows both the patient and his/her healthcare provider to monitor whether the patient is maintaining his/her target weight. If the periodic weight measurements start to deviate from the maintenance line, then the program can be modified restarting the full program.

The weight control programs of this invention are designed with the aid of a physician or expert, the patient's medical data and history and an extensive patient profile obtained from answers to a patient questionnaire. The programs are patient specific and are designed to achieve a desired final or maintenance weight in a practical period of time. From past methods and method failures, we have learned that rapid changes are not indicative of a well managed weight control program. Rapid weight change generally cannot be maintained and is sometimes dangerous. Although each program is patient specific, the method also includes acquiring and analyzing patient medical data and history, patient profile data and patient compliance data so that better weight control programs and feedback protocols or procedures can be implemented.

The physician or expert developed weight control program, sometimes called the Rx, along with certain personal, medical and profile data are entered into and stored in a host computer. The weight control program and selected personal, medical and profile data are then transferred to the weight measuring and feedback apparatus, the automated surrogate or monitor. The apparatus includes at least five components: a stable scale having linear response and repeatability of ±0.1 lbs, a processing unit such as a microprocessor, a computer, an analog processing unit, or a digital processing unit running software sufficient to weigh, compared and generate audio, visual and/or audiovisual messages, a colored LED display, a translation/voice unit and data storage. Preferably, the apparatus also includes communication software and hardware for communication with the host computer via wired or wireless formats. The critical measurement in the entire process is a periodic, preferably daily, metabolic weight, i.e., a patient's weight naked on arising and after voiding according to a specific periodic schedule, preferably each morning.

After initialization and establishment of a baseline weight, the robot will display and issue psychologically correct and physiological based audio, visual or audiovisual messages to the patient based on the daily weight measurement and its difference with the derived target weight value for the same period. The display will also show the patient graphically the patient's actual compliance data versus the program target weight values so that the patient can see how closely he/she is following the program and trends. The messages are designed to help the patient modify her/his behavior based on the compliance data and a difference between the actual weight value and derivatives and the target weight value for that period. The message can include suggested changes in exercise and in diet, the two controllable parameters in the caloric equation given above. The message can also include words of encouragement and/or encouraging visual images.

After 3 days, a first actual trend point is generated and displayed and with each additional daily weighing a new trend point is generated. From these trend points, a tentative actual trend plot can be generated and compared to the desired trend. Based on the comparison, the feedback apparatus issues verbal and/or visual recommendations for modulation of diet and activity based on the patient's stored data, e.g., if the patients favored activity is walking and the comparison evidences insufficient weight loss, the feedback unit can recommend that the patient increase the amount of time spent walking and/or increase the rate of walking (walking speed), and/or the apparatus can recommend an equivalent reduction in certain foods the patient eats based on the patient profile data.

The patient will continue periodic weighings and react to the apparatuses suggested modifications to exercise and eating habits for several weeks, two or more. After this period, the apparatus can send a data file to the host computer so that the treating physician or expert can review the data and based on the review recommend a second visit and/or directly modification the program, which is transferred to the apparatus for implementation at the patient's home. Alternatively, the process can includes a second interview/examination with the treating physician or expert. The patient would either bring a removable data storage unit associated with the apparatus such as a data stick, CD, diskette or the like. Of course, if the domicile is equipped with a wired or wireless network, then the feedback/monitoring unit can be plugged into the network and periodically directly transfer information from the domiciled unit to the expert or host computer at the expert's location or at an expert monitoring server facility. In the second interview/examination, the treating physician or expert will perform a brief followup interview, a brief medical evaluation of patient and a review of the data file stored in the apparatus. Any desired changes to the program will be entered into the host computer and transferred either to the apparatus or to a removable storage unit for implementation of the changes. The process using the weighing/feedback apparatus or domicile monitoring unit will be indefinitely continued, when a desired maintenance weight is reached; the apparatus will continue to monitor patient weighings to insure that no change in weight or significant deviation from the Rx zero slope trend line. Thus, the program aids the patient over an extended period of time, preferably, for the rest of his/her life to ensure maintenance of a target weight. Once stability has been achieved, the apparatus can be either set to a maintenance format, where the apparatus still measures and stores the weight can be replaced by a less sophisticated apparatus, which only includes a simple display, monitoring hardware and software and communication hardware and software. Of course, the program can be reimplemented if upon review the treating physician or expert detects a departure from the maintenance weight over a statistically significant period. Moreover, the data transferred to the host computer for each monitored programs in its active or maintenance phase will be used to better tailor the patient interview/examination process, to formulated patient categories, and to develop improved weight control programs utilizing audio and/or visual feedback formats. Of course, for visually and hearing impaired patients, tactile based feedback protocols can be used.

The system of this invention is based on established physical and physiological principles beginning with the principle of the conservation of energy, expressed in weight control context as the balance between energy intake and expenditure measured in “large” vs. “small” calories, cal v. cal. In any system the sum of energy input to the system must equal the sum of the energy output and energy stored. In the human body, there are also internal energy stores, primarily fat, and except in the case of muscle building exercises or starvation, any difference between calories input in food and output in body activity, internal plus external, will be reflected in gain or loss of fat and of fat associated weight.

With growth or muscle hypertrophy through exercise a portion of the diet is transformed into tissue also producing increased weight. The present invention relates to a unique weight monitoring and feedback system that measures and derives weight changes with an unprecedented resolution allowing the most rapid and exact practical control to date. Such resolution also allows previously unavailable self-education of the subject as it comes to weight control, weight fluctuations, and weight loss or gain trends.

This capacity is incorporated in a process which maximizes support of the patient in their use of the monitor to control their weight by several unique features including: (1) using the authority of a physician or other expert at all points of the program, (2) an extension of this authority by means of a monitor generated voice recommendation based on each day's weight determination, (3) minimizing patient time and effort by limiting the daily process of monitoring, (4) evaluation and recommendations to 1-2 minutes, (5) minimizing disruption of the patient's usual life style by making realistic, and (6) small and physiologically sound daily changes over realistic periods of time through recommendations of small daily adjustments of patient's usual diet and activity. These recommendations are based on each subject's psychological, dietary and activity profile, i.e., the program is individually tailored to the patient. Maintenance of desired weight, a major point of failure of existing programs, is made possible by continued simple monitoring.

While the present program is designed for normal patients under expert control, it can be modified to a range of situations from morbidly obese in institutional situations to the do-it-yourself individual.

Correct measurement of weight changes is currently the basis of any reasonable weight control program. Body weight varies continuously in the short term, both diurnally and transiently, and in a weight control program, long term changes must be meaningfully extracted from this noise. Failure to do this is a major problem in all current programs.

It is generally accepted by knowledgeable workers that the most reproducible time to measure human weight is just after sleep and voiding, but before any food intake. Also this “basal” weight must be obtained, confounding factors such as clothes. This is an important procedural point incorporated in the system.

However, normally there are daily transient variations of up to several pounds, usually produced by variations in diet, environment and activity. At the same time prescribed weight loss should be on the order of 0.1 lbs per day so methods of extracting data with such resolution must be incorporated in the system.

These requirements are met by a combination of mensuration technology and data processing. Resolution and long term accuracy of ±0.1 pounds is common practice in many scale applications, which typically consist of a means of transferring weight forces on a platform to a cantilever beam or other mechanical element, where force is converted to displacement which is measured by a strain gauge or other such device. Scale performance should not be confused with claims often made for consumer scales. A class 3 scale certified for use in trade is generally required. The electrical output is digitized, processed and displayed or transmitted. Initial processing of this crude weight data may include corrections for zero errors, gravitational variations, etc. and may be processed by signal averaging with the result that body weight may be routinely and reliably obtained with ±0.1 lbs stable resolution. Such techniques are employed as a component of the system of this invention.

Measurement of weight is the accepted standard for any control program, and properly done, avoids the difficulty of dealing with multiple complexities such as variations in energy conversion, diet, activity, basal metabolism, genotype, etc. by providing a single measure of the results of the combination of such controlling factors. Moreover, it can rapidly and unequivocally validate the effects of any intervention by these factors and provide criteria for changes in diet and activity to achieve a desired weight goal. However, obtaining weight data in usable form must be done with rigor and precision. Adequate weight measurement is the point of departure for the present system from prior art systems.

Currently most professional scales have a resolution and stability of ±¼ lb, although ±0.1 lb scales are occasionally seen in clinical use. However the accuracy of such scales is wasted because of time and technique; time of measurement is typically random as is the weight of clothing worn by the patient. This often results in errors of pounds. A brief resume of other weighing problems follows.

Normal weight has a diurnal pattern of minimum weight after the usual sleep period with various gains and losses throughout the day. The most reproducible or basal weight occurs on rising from a normal sleep period, after voiding and before intake and without clothes. This is a standard procedure in the The present system program. However, day to day variations of up to a pound in basal weights are usual where diet and activity vary, e.g., a variation in body fluid volume through varying intake and loss is a major source of transient weight change but such changes in weight are greatly reduced by corrective fluid volume changes by urinary excretion during sleep.

Single large meal can transiently add a pound or more, while jogging on a hot day can lose as much or more. Since a typical desired weight loss is 0.1 lb/day, means of extracting this order of significant change from the “noise” of daily fluctuations must be used. At this point, comment on frequency of weighing is in order. Ideally a continuous measure of weight throughout the day would allow the most rapid changes in diet and activity for control purposes but this is not practical.

Conversely, in programs with the usual errors in weighing, a considerable time is required for changes large enough to be seen to occur, typically a week. Even then changes may be confused by variations in the usual weighing process. Also most programs aim for large losses for advertising. This combination can cause several problems including: the patient has an often rigorous program to follow without support for a week, at which time actual change may be diminished or obscured by a positive error or a much greater change may be apparently produced by a negative error. In either case discouragement, confusion and inappropriate changes in the program may result.

A unique part of this invention is extraction of meaningful data from daily basal weights. This is accomplished by processing the weight data in a digital processing unit in the domicile monitoring unit. A number of statistical programs can be employed for this purpose, but a simple 3 point sliding mean average has been shown to be adequate. The initialization process includes the patient or user making 3 basal weight measurements under the standard conditions described above so that less than a 0.1% difference is obtained. This value is taken as the initial weight point, which is then stored in memory and displayed on the display unit.

Weights are taken on the following two days and the three days results are averaged. This average then becomes the mean starting weight and origin of two lines with slopes of actual weight and desired change in pounds/day.

The present system has chosen daily sampling as the shortest practical period for control purposes. However day to day variation in basal weight could also lead to frustration or inappropriately large changes in diet and activity, so several processes are used to sense and achieve the resolution needed to make the small changes desired, as follows. The desired weight change is a small continuous decrease, typically 0.1 lb/day and this is chosen as a target. The present system goal is to provide valid information daily to allow the patient or user to produce actual changes of this magnitude. A meaningful actual trend can be obtained from daily basal weights by statistical analysis of weights over a number of days, albeit with a time lag. A method successfully used to obtain useful data in the present system is a three-day sliding average in which each day the mean of the actual weight, plus that of the two previous days is computed. This produces a much more representative value that can be meaningfully compared to the desired weight.

Daily verbal recommendations are made to the patient or user by the monitor based on a combination of: daily weight difference from desired weight curve value, the difference between today's and the last previous weight, the difference between the calculated mean and desired weight, and the slope of the actual mean trend line versus slope of the desired loss trend line. These data are computed and usually displayed to the patient in graphic form and combined with stored patient data to produce a vocal recommendation for any required change in daily activity. This process is sensitive enough to allow implementation of the small regular changes that are a major element of the present system program.

Spot weights may also be taken at any time and while not recorded, are a valuable source of patient education. Resolution of the scale is such that the effects of small intakes or of various activities may be seen by, before and after measurements.

A visual display of current values is an important part of patient feedback and self education. Colored symbols are used to enhance the feedback. Currently, blue or green is used to denote daily weight, red points and connecting lines denote mean weight values and a solid black line represents the line of desired weights and rate of loss or gain.

Daily basal weights show short term effects from transient changes in diet, activity, etc. This is important for patient self education. The value of high resolution is further enhanced by digital output, but not graphic output, of spot weights at any time of the day after the basal weight measurement for that day has been taken. This feature allows the patient to see effects of activities as they occur during a day. Scale resolution allows the effects of even mild exercise or a small snack to be seen by before and after spot weight measurements.

Once means of obtaining reliable basal weight changes over short periods have been established a process of optimally effecting weight changes must be designed. This is a complex problem, for no two people are exactly alike, and in the typical situation where virtually any kind and quantity of food is available at very low energy cost to the patient, the outcome of any voluntary program is ultimately psychologically determined.

The program of this invention is designed to bolster any patient motivation to change weight wherever possible by numerous means. This process begins by initially placing the patient under the authority of a physician or other professional expert.

Crucial to success is providing a program which “fits” the individual and provokes minimal stress and resistance, so the first step is to characterize the patient, beginning with their pertinent physiological characteristics.

A synthesized voice will provide daily suggestions to the patient as an extension of the expert, for authority and for support, in addition to conveying information. The phrasing and nature of this voice depends not only on the information to be conveyed but also on the psychology of the subject. A drill instructor's voice my work for a few, but can more often be counterproductive. Initial determination of the patient's relevant psychology will be performed by a questionnaire and may be modified by expert patient evaluations, both the initial evaluation session and especially during subsequent evaluation sessions when sufficient daily weight data is available from the domicile monitoring unit.

A second essential set of information needed to generate a weight control program of this invention involves the determination of a patient's dietary and activity habits. For example, it would be ridiculous for the synthesized audio message to suggest decreasing cereal consumption at breakfast, if the patient does not eat cereal for breakfast, or that a subject would not be asked to increase walking distance, if the patient suffered from osteoarthritis. The usual type and amount of a patient's food intake, a dietary profile, and the type and duration of physical activity, an activity profile, will also be obtained by questionnaire. Any physical limitations will be determined by questionnaire. Questionnaire data will be verified or expanded upon during the expert evaluations. History and a few critical physical measurements will be taken for reference.

Although this system will allow virtually any detail of psychological, dietary and activity attributes or characteristics to be incorporated, emphasis is on efficiency and minimum patient impact, so for physiological fitting of the program to the patient, a limited number of personality types will be matched to a few synthesized voice programs, i.e., the synthesized voice usually will be limited in response.

Normal dietary ranges of the patient will be entered as quantities and types of food likely to be consumed at a given time. This information will be stored in the expert's computer, and caloric values automatically derived from stored lookup tables, preparatory to transfer to the patient's domicile monitoring unit. The same process will be applied with time and type of activity. The expert computer will estimate energy costs of patient activity scaled to the individual patient. After patient-expert consultation, any patient limitations will be entered into the program (e.g., osteoarthritis, etc.). Once all preliminary data is entered, a weight gain or loss goal will be created and stored in the expert computer data file and an initial personalized weight control program, to be loaded into the memory of the domicile monitoring unit, will be generated. Once generated, the personalized program is loaded onto the domicile monitoring unit.

### DETAILED DESCRIPTION OF THE DRAWINGS

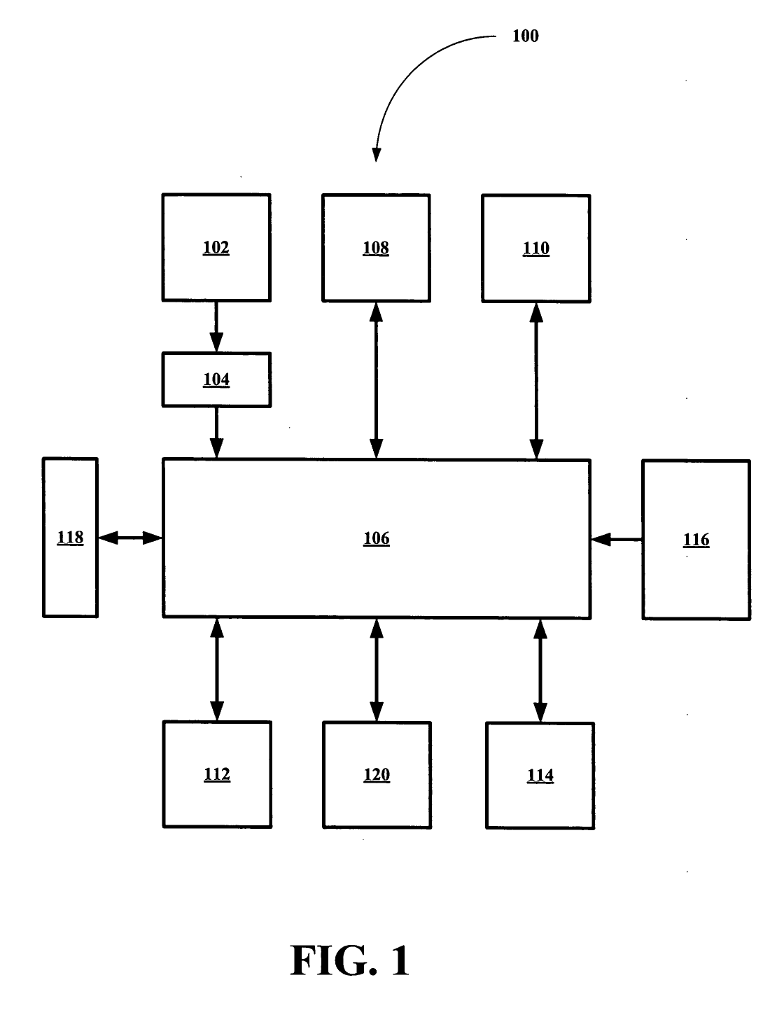

Referring now to FIG. 1, a block diagram of a preferred embodiment of a monitoring apparatus, generally 100, is shown to include a digital scale 102 having an accuracy of ±0.1 lb. The apparatus 100 also includes an A/D converter 104. Electrical changes in the weight element of the scale 102 will be detected and digitized in the A/D converter 104. The digitized scale reading is then processed and stored in a DPU 106, which is preferably a single board computer. The DPU 106 comprises a digital processor and includes a memory 108 having encoded thereon or therein a patient weight control program along with necessary software including an operating system, communication hardware and software, etc. The DPU 106 also includes a removable memory stick port 109 supporting a memory stick 110. The DPU 106 also includes a display 112 an audio output unit 114 having a speaker (e.g., a voice synthesizer), a power supply 116 and a clock 118. Conventional keyboards, etc, are required, but the apparatus 100 can include optional keyboard type data entry devices. The display 112 may include one or a plurality of buttons, screen selectors, or switches for invoking a major control function such as program reset, etc. If multiple patients are to use the same apparatus 100, then the apparatus 100 can include a patient identification subsystem. The subsystem is generally incorporated into the audio output unit 114 and includes a microphone which is designed to receive a voice input and the DPU 106 includes voice recognition software to identify the voice input. Once identified, the DPU 106 configures the apparatus 100 for the identified user. Alternatively, the apparatus 100 can include a key pad associated with the display unit 112 for entering a unique code. In yet another alternative, the apparatus 100 can include a bar code reader or a magnetic strip reader or thumb print identification device, or a similar device for discriminating between users. The DPU 106 would in turn have the software necessary for receiving the user discrimination data and configuring the apparatus 100 for the identified user. The apparatus 100 can also include wired or wireless communication hardware and software 120 for connecting to the internet for downloading weight control programs and uploading data to the host computer for periodic review by the healthcare provider.

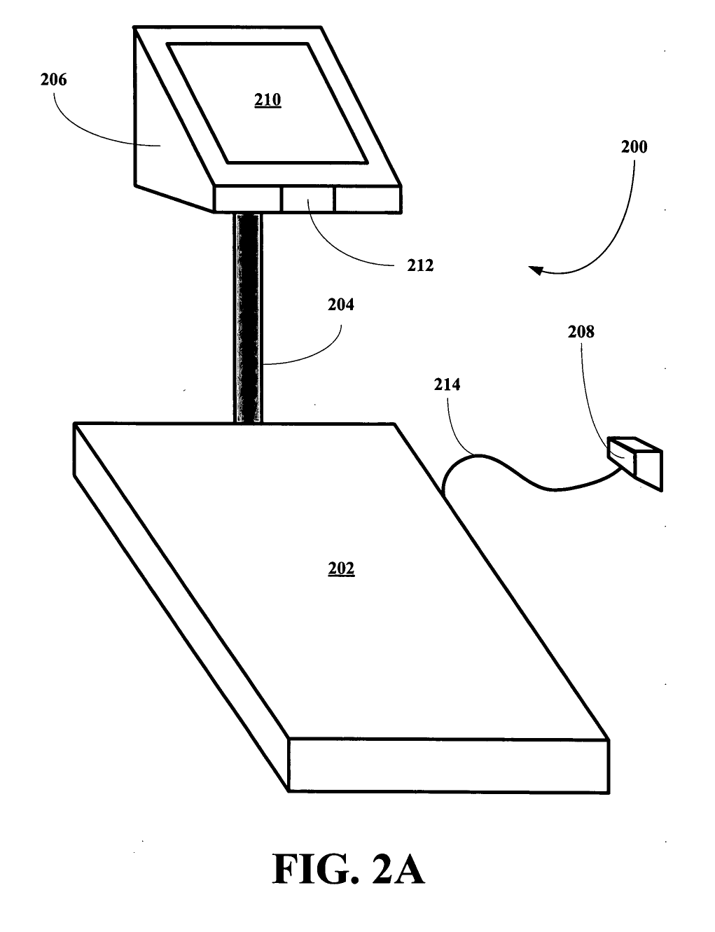

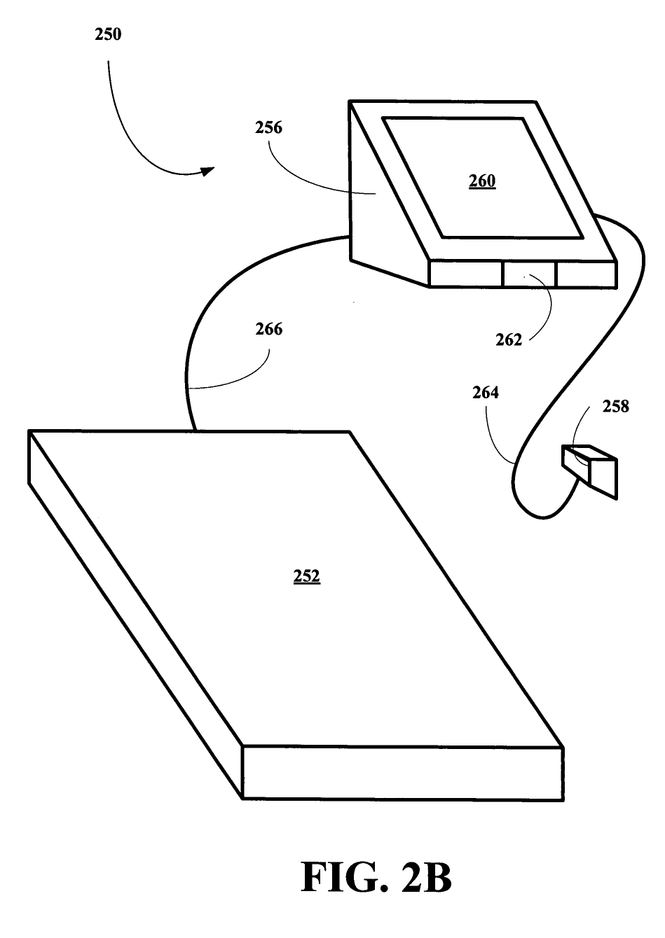

Referring now to FIG. 2A, a preferred embodiment of the apparatus of FIG. 1, generally 200, is shown to be of an unitary construction including a scale 202, a poll 204 supporting a display and feedback unit 206, and a power conditioner 208. The display and feedback unit 206 includes a display 210 and a speaker 212. The poll 204 includes an electrical conduit (not shown) connecting the scale 202 to the unit 206 and designed to provide power to the unit 206 and electrical communication between the scale 202 and the unit 206. The power conditioner 208 is connected to the scale 202 via a power cable 214.

Referring now to FIG. 2B, another preferred embodiment of the apparatus of FIG. 1, generally 250, is shown include a separate scale 252, a separate display and feedback unit 256 and a power conditioner 258. The display and feedback unit 256 includes a display 260 and a speaker 262. The power conditioner 258 is connected to the scale 252 via a power cable 264. The scale 252 is connected to the unit 254 via a electrical and communication conduit 266.

Operation is illustrated by the following usual sequence of start up and use. The monitoring unit operates continuously and various conventional automatic power up/down modes may be incorporated. This unit is programmed to accept basal weights only within a selected period of time, the basal weighing window, which generally is set to correspond to the patient's morning start time. For example, for most people, the weighing window will be between 6:00 am and 9:00 am. The system accepts only one basal weighing per day.

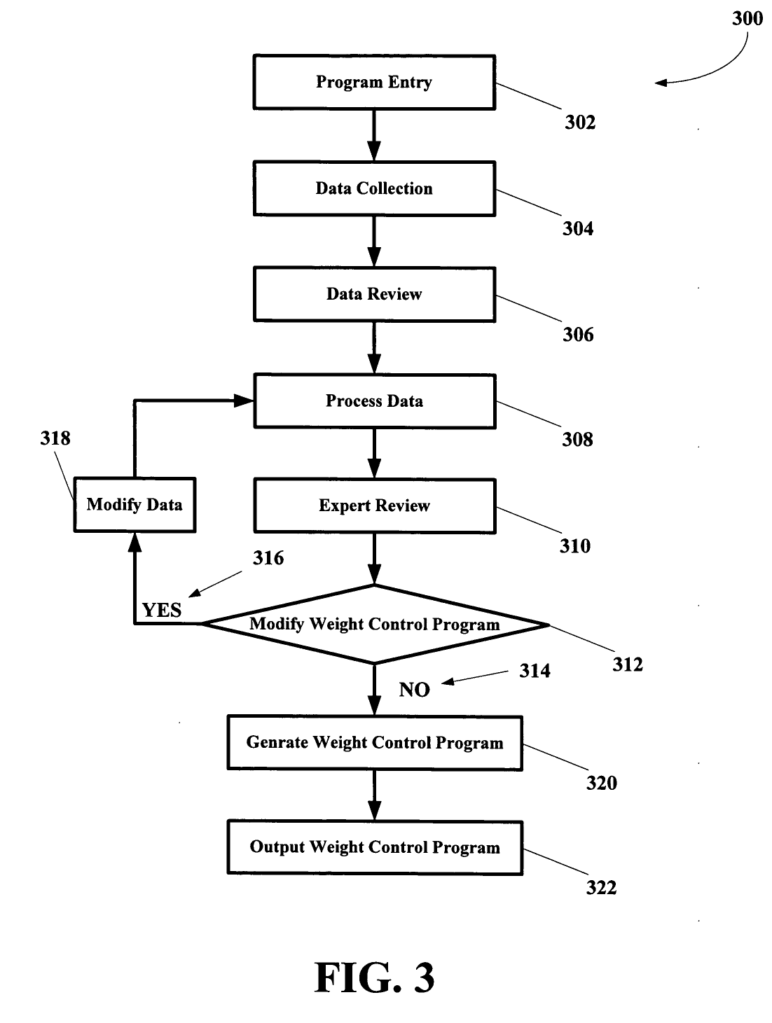

Referring now to FIG. 3, a flow chart, generally 300, of a preferred process of this invention for generating a weight control program is shown to include an entry step 302, where a patient is entered into the program. One aspect of the invention is it is designed to support as many patient types and motivations as possible. The program is designed to allow a patient entering the program to include existing, safe diet and/or activity or routine, which can be incorporated into the feedback the patient receives from the monitoring unit during routine usage. For patients in denial of a problem or experiencing other mechanisms of self denial, a higher level monitoring can be added to the created weight control program. If the patient is group oriented, especially family, the monitor can easily accommodate large groups. Of course, certain patients may not be candidates for this type of a program. Such patients may be screened out at the time of entry or may not respond to the program regardless of the level of interactivity.

After the patient is admitted into the program, data about the patent is gathered in a data collection step 304. Although the data can be gathered by question-answer sessions with data entering personnel entering the data into a host computer, preferably patient data is collected using preformatted questionnaires, which the patient completes. The data contained on the questionnaires are scanned into data structures on the host computer. Psychological questionnaires are designed to determine the nature, phraseology and tone of the daily feedback messages most suitable for the patient. For example, the expert's voice may be used instead of the usual synthesized voice. Questions on diet will document usual types and amounts of food eaten by the patient on a routine basis. Activity questions will cover types and amount of current activities from sleep to work, exercise and games, etc.

After data collection, the healthcare provider and the patient set down to review data, to go over medical history and if necessary, the healthcare provider gives the patient a physical in a data review step 306. The data review step 306 generally begin with a review of the questionnaire and answers thereto insure validity, followed by a review of health and condition with particular attention to medications and problems relevant to the implementation of a successful program, e.g., musculo-skeletal and cardiovascular system. A limited exam is done to confirm any historical problems and to discover any problems relevant to implementing a program of this invention. This physical will normally include a few baseline measures such as vital signs, blood indices and relevant hormonal values plus estimate of body fat, stature and a few key girths.

After data review, the data is input into a host computer in an input data step 308, where the data is processed and stored for later inclusion in the weight control program. Using caloric values of food and exercise derived from lookup tables and data concerning the patient's diet and exercise habits, caloric input and output factors are approximated. The program can either suggest a desired final weight goal or the patient in the questionnaires could have requested a final weight goal. Based on the final weight goal, the program also generates patient tailored audio output files, where the files includes statements designed to suggest, recommend and encourage successfully implementation and adherence to the final weight control program.

Next, the patient meets with the expert who reviews the proposed weight control program with the patient in an expert review step 310. The expert also discusses any outstanding issues or concerns. During the discussions, the patient and expert can either accepted the proposed weight control program or modify the weight control program in a conditional step 312. The modifying process can continue until, a program is arrived at the both the patient and expert agree is achievable and well-suited to the patient's psychological and physiological conditions. If no modification or nor further modification is needed to the weight control, then the process proceeds along a NO branch 314, otherwise, the process proceeds along a YES branch 316 to a modify weight control step 318 and back to the process data step 308.

At this point, the process generates a data file containing the weight control program in a generate weight control programs step 320. Once generated, the weight control program in a transfer step 322 can either be transferred directly to the domicile unit 100 or to a memory stick, which the patient can then used to transfer the program to the domicile unit 100 or, if the domicile unit 100 is set up with an internet connection, then the weight control program can be transmitted over the internet to the domicile unit 100. The weight control program includes a weight goal, a weight loss/gain trend line having a desired slope and a duration over which the patient is required to attained the weigh goal.

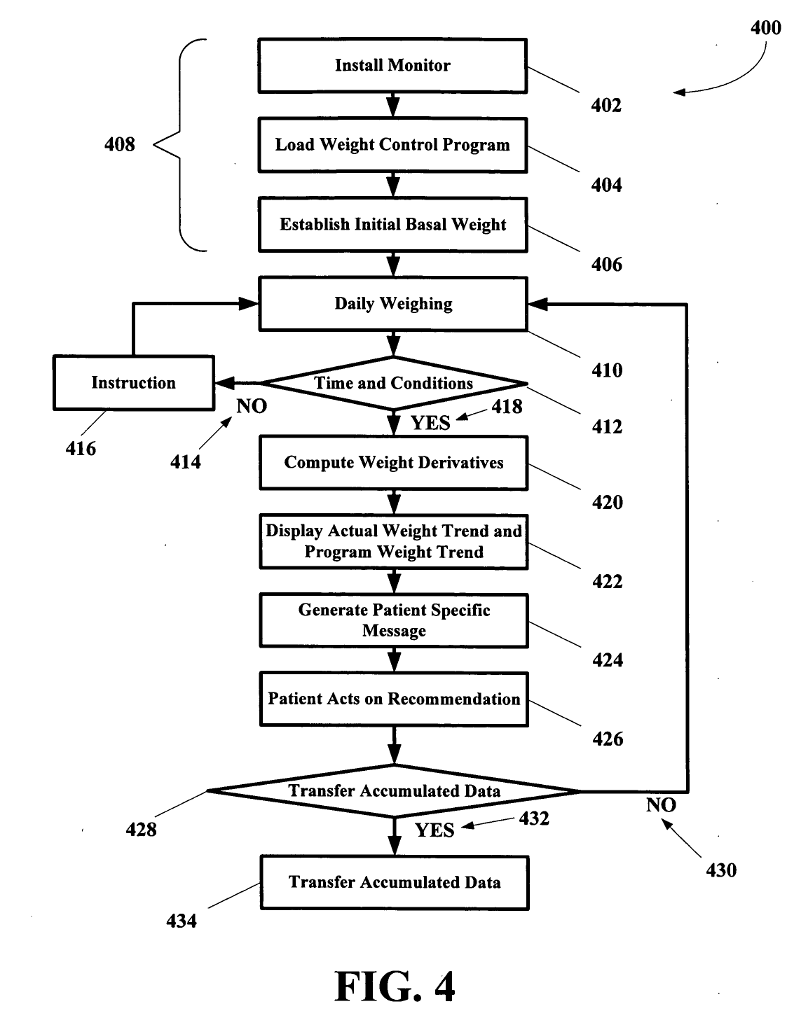

Referring now to FIG. 4, a flow chart, generally 400, of a preferred process of this invention for monitoring patient adherence to the weight control program is shown to include an install monitor step 402, a load weight control program step 404 and an establish an initial basal weight step 406. These three steps represent the monitor initialization process 408. When the monitor is first brought to a patient's domicile. The unit is plugged in. Once plugged, the unit can either require inputting of the weight control program or simply loads the weight control program that was already transferred to the unit before the unit was brought to the patient's domicile. If the weight control program is not already loaded, then the patient can either load the program via a memory stick including the program or via attaching the unit to the internet and the unit will download the program. Once the unit is on and the program installed, then the patient steps on and off the scale a sufficient number of times so that 3 consecutive weighings difference by no more the 0.1 lb.

Once the monitor unit has been initialized, the patient or user can weigh himself/herself at any time in daily weighing step 410. After a weighing occurs, the program determines whether the weighing should be included in the daily weighing data table stored in memory of the unit in a time and conditions conditional step 412. The conditional step 412 checks the date, time, and weight value of the weighing. If the date, time and/or weight value is outside of the date, time and weight tolerance limits of the program, the program will not accept the weight as a daily weight and the control is transferred along a NO branch 414 to an instruction step 416 and back to the daily weighing step 410. In the instruction step 416 instructions are generated for the patient on the proper time and conditions obtaining a proper daily weight. The instructions include that the daily weight need to be taken during a specific time range, in the nude and after voiding, but prior to the ingestion of any liquid or solid matter. If the weight is within the time and condition values set in the program, control is transferred along a YES branch 418 to a compute weight derivatives step 420. In the compute step 420, the daily weight is used to determine a mean average value from the current weight and the previous two weights. Of course, in the first two days of use of the monitor, the daily weights are accumulated, but are not used to produce a mean weight average value based on three consecutive value for use in trend plotting, because the trend plotting of this invention is based on a sliding mean value trend evaluation process. The monitor is programmed to accept basal weights only if obtained within a set time frame, e.g., 08:00±2 hours, and after initialization, only within a set weight range, e.g., ±3% of the daily previous weight.

Once the weight derivatives are computed, the results are displayed on the display showing the actual weight data and the weight control data in a display step 422. The data is generally displayed as points for the daily weights, a trend line for the weight control program or target weight trend values, and a trend line for the sliding mean average. Simultaneous with, prior to or subsequent to the display of the weight data, the program generates a message to the patient in a generate message step 424. The message generally comprises recommendations specific to the patient based on the patient specific data transferred to the monitor unit with the weight control program and based on the weight data computed in the compute step 420.

After the patient has seen the weight trend data on and has heard the message from the monitor unit, the patient acts on the recommendations in an act on recommendation step 426. The process is continued for a specified number of days determined by a conditional step 428. If the number of days is not yet reached, then control is transferred along a NO branch 430 back to the daily weighing step 410. If the number of days has been reached, then control is transferred along a YES branch 432 to a transfer accumulated data step 434, where the accumulated data is either transferred to a memory stick for transportation to the patient's healthcare provider for transfer to the host computer or transferred to the host via the internet.

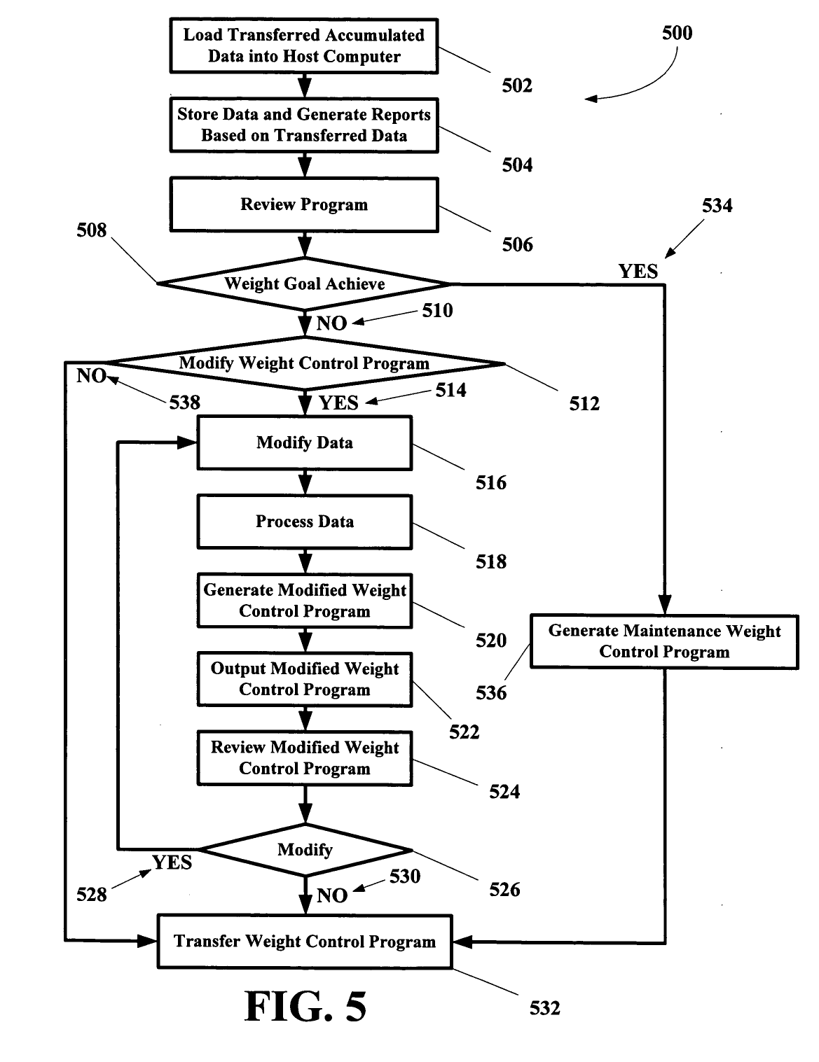

Referring now to FIG. 5, a flow chart, generally 500, of a preferred process of this invention for reviewing a patient's adherence to the weight control program is shown to include a load step 502 followed by an store data and generate report step 504. After the data has been loaded, stored and reports generated, the data and reports are review by the patient and the patient's healthcare in a review program step 506. The program also includes an achieve weight goal step 508, where the review determines whether the patient has achieved his/her weight goal. If the weight goal has not been achieved, then control is transferred along a NO branch 510 to a modify program step 512, where a decision is made to leave the program as is or to modify the weight control program based on the actual weight data, the target weight data and the patient's adherence to the weight control program. If the program needs modification, then control is transferred along a YES branch 514 to a modify data step 516, where patient data is modified and fed to the host computer. The host computer then processes the modified data in a process step 518. After data processing, the program generates a modified weight control program in a generate step 520. After generation, the modified weight control program is output in an output step 522. The modified weight control program is then reviewed by the patient and the healthcare provider to determine whether additional modification is needed in a review step 524. If additional modification is needed, then, in a second modify step 526, control is transferred along a YES branch 528 back to the modify data step 516. If no additional modification is needed, then control is transferred along a NO branch 530 to a transfer weight control program step 532, where the weight control program is transferred either to a memory stick for later transfer to the domicile unit or over the internet to the domicile unit. If the weight goal in step 508 has been achieved, then control is transferred along a YES branch 534 to a generate maintenance weight control program step 536, where the program modifies the program to includes a trend line having a slope of zero at the maintenance weight. Control is then transferred to the transfer step 532. If after the review step 506, the weight goal has not been achieved and the healthcare provider does not modify the existing weight control program, then control is transferred along a NO branch 538 to the transfer step 532.

After an appropriate time, typically several weeks, or earlier if there is a problem, the patient or user transfers the collected data to a memory stick or other transfer device or uploads the collected data via the internet to the host computer. The patient or user than schedules an appointment with his/her healthcare provider for a meeting to review the collected data. The review begins with an evaluation of program to date. On the first review, a followup physical exam is generally not done. If the collected data is on a portable memory device, then the healthcare provider transfers the collected data to the host computer. The host computer then generates a display of the data to date for review by patient and expert. If they decide any revision is needed to the program, the revisions are input to the host computer, which will incorporate the changes in a revised patient weight control program. If the program is revised, it will be transferred to a memory device or other portable storage device for later transfer to the domicile unit or downloaded via the internet to the domicile unit. With later reviews in which significant change may have occurred, the healthcare provider may give the patient a follow up physical examine and any significant changes in physical or physiological data is transferred to the host computer and if appropriate incorporated in the monitor program.

The patient continues the weight control program performing daily weighings and following program generate recommendations, until the desired weight is reached.

Alternatively, after the patient has continued to weight control program performing daily weighings and following program generated recommendations, until the desired weight is reached. The review will include a physical examination and the findings will be stored in the host computer. The new data will be considered in the review, and the weight control program will be changed to a no weight change weight control program. This new program will be transferred to a memory stick or other portable memory device for subsequent transfer to the monitor or the new program will be downloaded to the monitor via the internet. The patient will continue the weight loss program at a maintenance level unless the collected data show a change from the zero slope maintenance weight. If such as change occurs, then the healthcare provide will modify the weight control program after a review and a new program will be initiated.

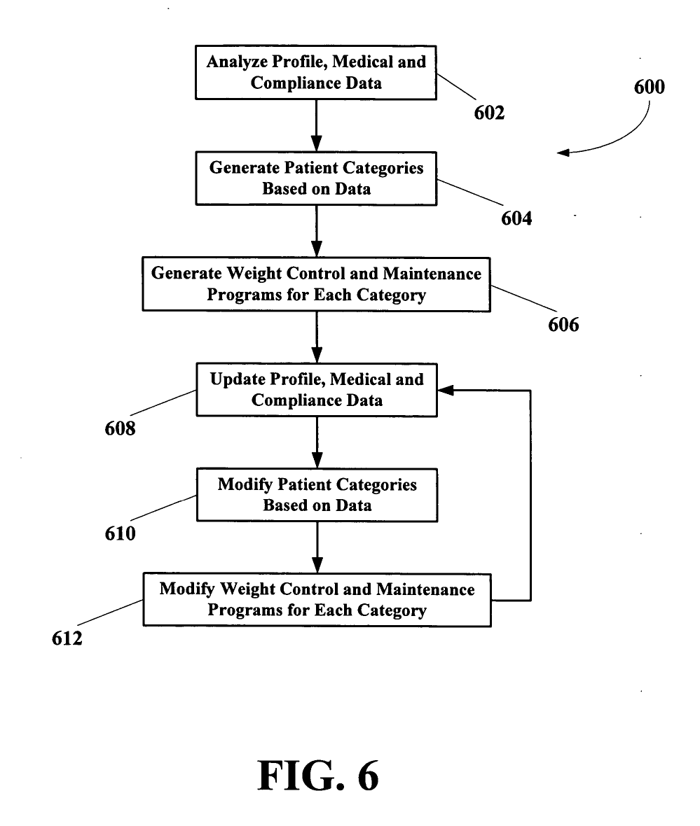

Referring now to FIG. 6, a conceptual flow chart, generally 600, of a preferred weight control analyzes and development program is shown. The chart 600 includes an analyze profile, medical and compliance data step 602. From that analysis, patient categories are developed in a generate step 604. For each category, weight control and weight maintenance programs are generated, modified, and optimized in a second generate step 606. As new data is added to the host computer in an update step 608, modifications are made to the patient categories in a modify step 610 and modifications are made to the weight control and maintenance programs for each category in a second modify step 612. This process in continued until no more modifications or categories are required in the program, indicating that optimal programs have been developed. Moreover, the data and analysis can be used to modify the questions asked the patient in the physician or expert interview process and/or to modify the type of medical and personal data obtained in the interviews and examinations. The analysis and modification processes are designed to product better programs tailored for each individual and to better address individual motivational formats and encouragement formats.

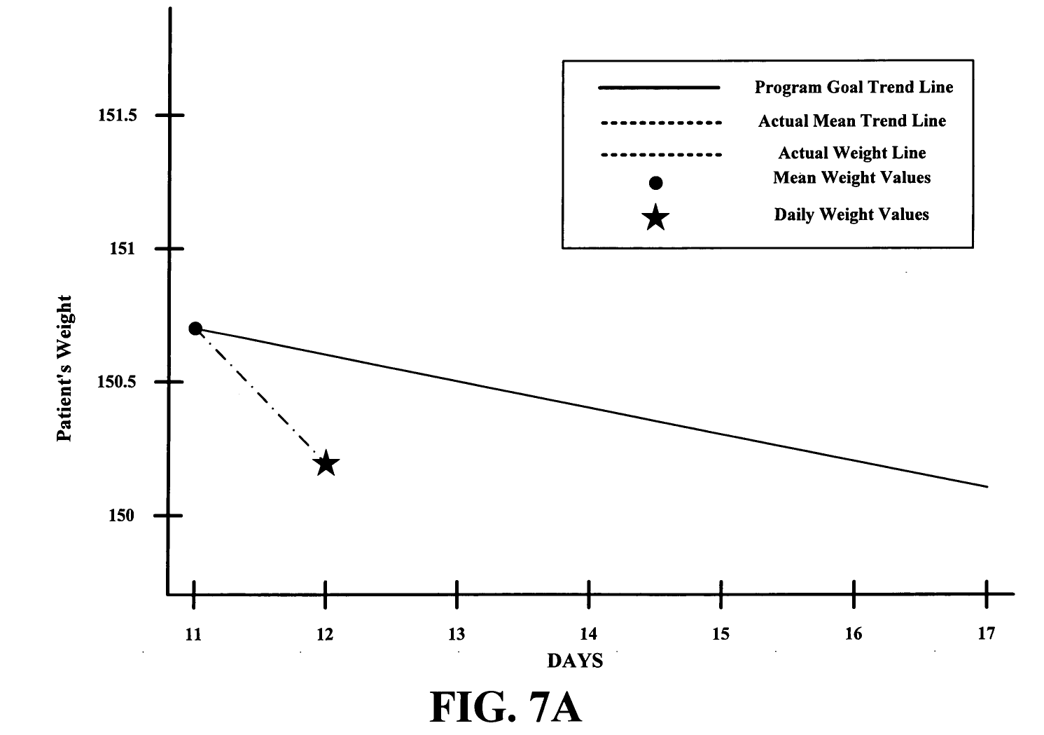

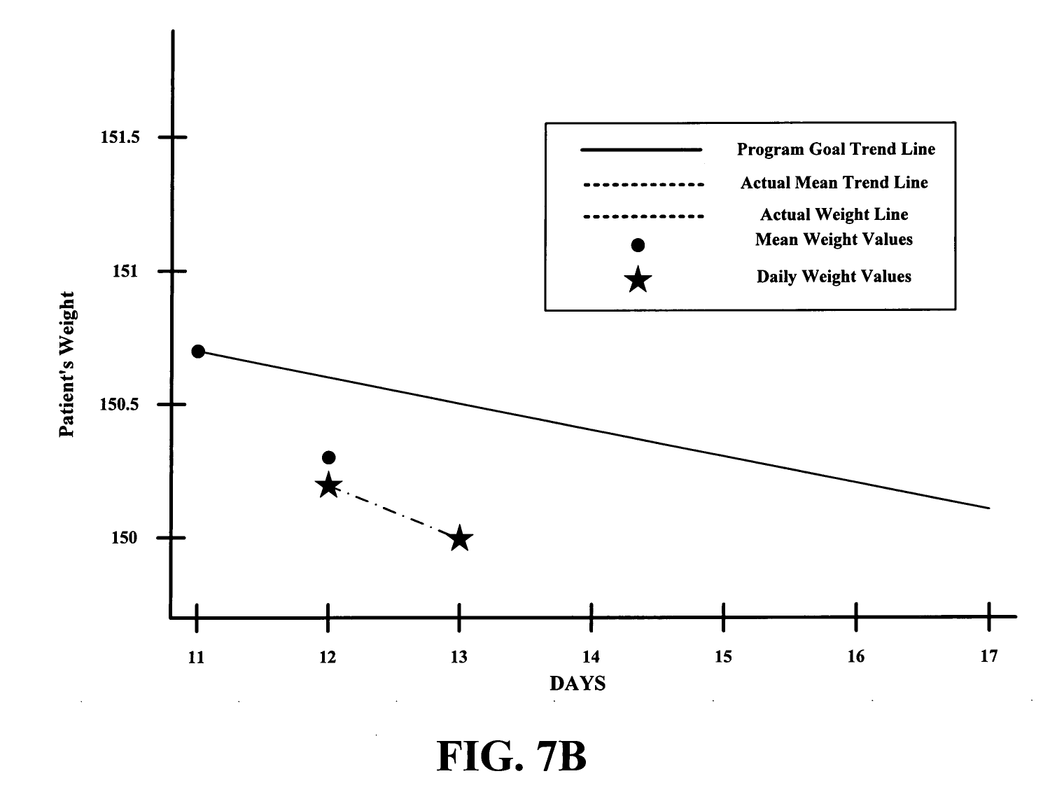

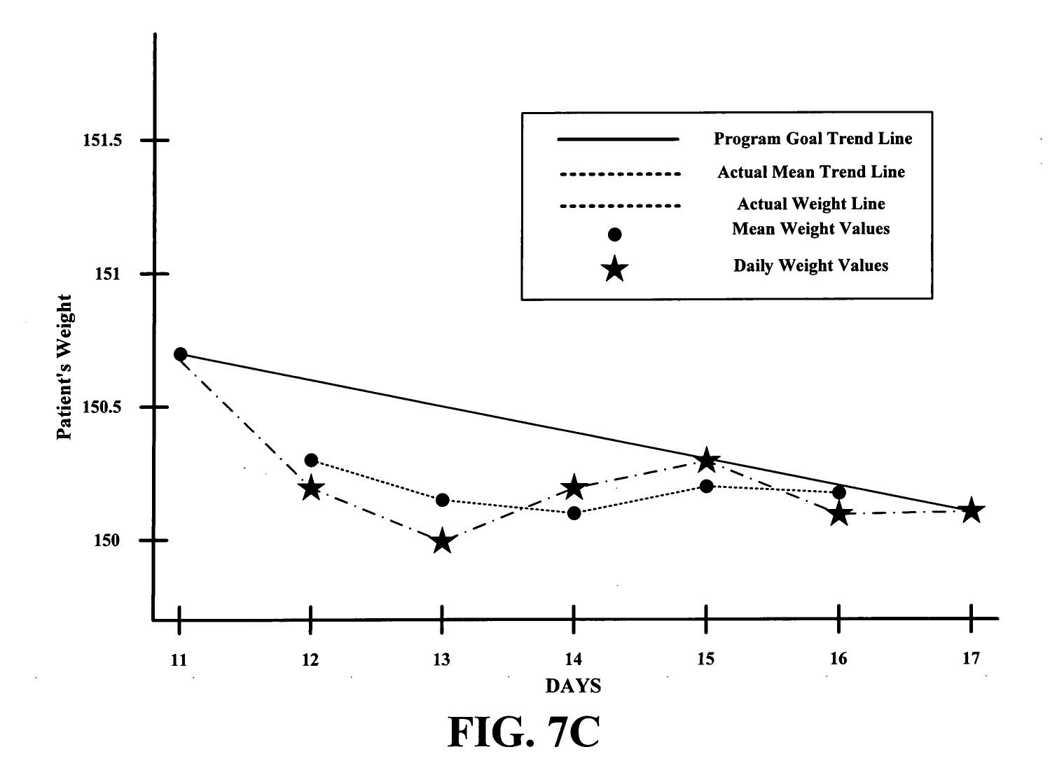

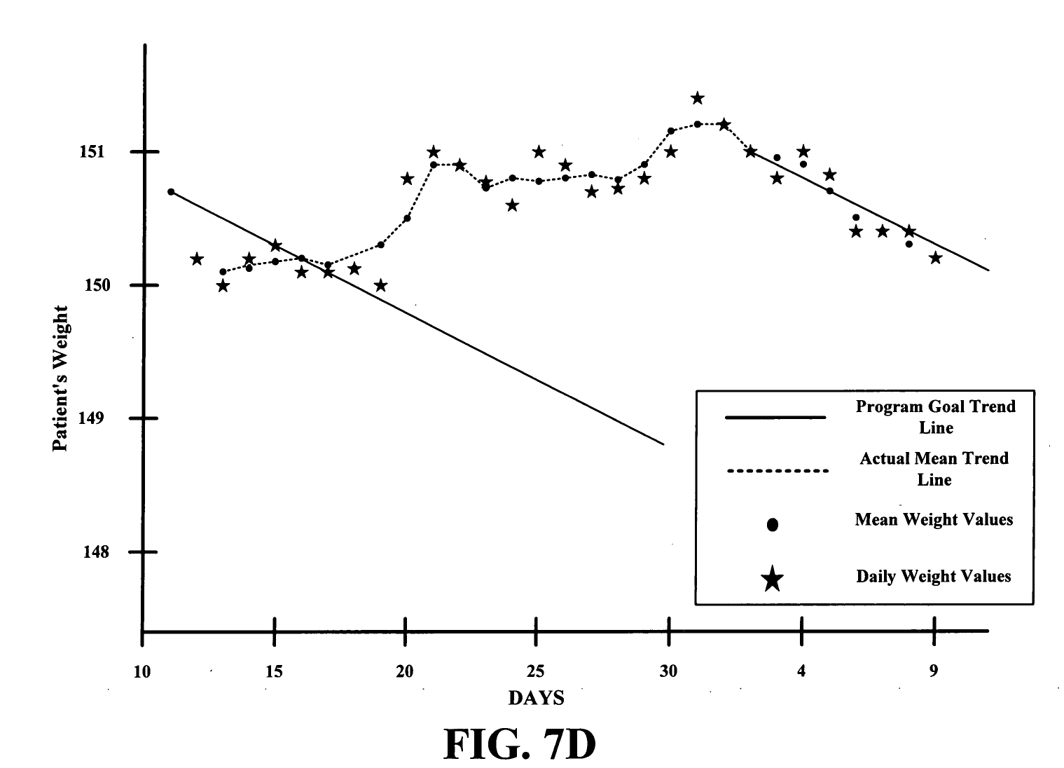

Referring now to FIGS. 7A-D, an illustration of accumulated daily weighing data and the computation of the sliding mean averages and the sliding mean average trend line relative to the weight control trend line. Looking at FIGS. 7A, on the first day, the patient step on and off the scale a sufficient number of times to obtain three consecutive weights that differ by ±0.1 lb within the daily weight window, in the nude, after voiding and before ingestion of liquids or solids. This point is then displayed as the solid circle and the weight control trend line is generated with this circle as its origin. The next day's basal weight is added to the display as shown in FIG. 7A. Depending on the location of the second day weighing, the program may as in the case shown in FIG. 7A, generate a message such as “Although it is too early to be sure, your weight appears to have fallen even more rapidly than planned. Keep doing the same thing and tomorrow we can tell much more”. The program may optionally connect the actual daily weights with a line to show not only the program trend line, but the daily weight trend line and the sliding average trend line after sufficient points have been generated to display the average trend line.

Looking at FIG. 7B, on the third day, the daily weighing is added to the plot and the first three day sliding average is calculated and added to display as another distinctive point. The first sliding average point is placed on the day axis at a midpoint of the three daily weighings used to compute the average. This point represents a best approximation of the actual basal weight and is combined with the current actual day's weight, differences between the mean and actual basal weights and desired weights, and rate of change of previous day's to the current day's basal weight and other relationships between the data are calculated and stored.

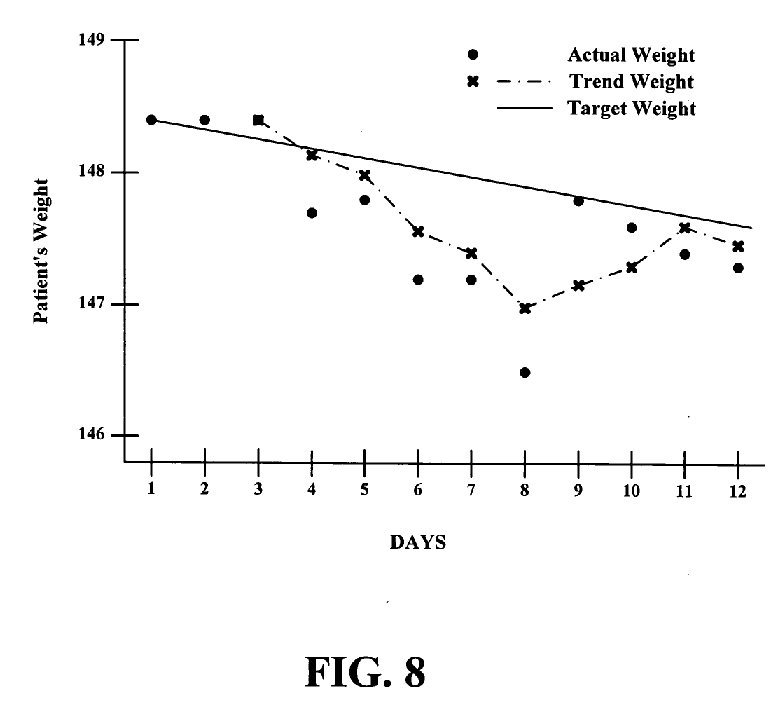

Looking at FIG. 7C, a plot of sample data collected over a 7 day period is shown. It can be seen that the patient started off well. But by the seventh day, the actual mean value plot is showing This provides individual privacy for multiple users. At this time a patient ID will be assigned such as CBI, CB2, etc. recommend, FIG. 2, C, a modulation of diet or activity to produce a rate of weight change that will intersect with the line of desired weight change in a reasonable time, e.g. with the situation shown on FIG. 7, a message might be “you are losing weight faster than planned and might be more comfortable if you added X food (X is an amount calculated from several of the subject's recorded favorite foods) or reduced Y activity by Z. (Z is a calculated reduction in some known activity, Y). This will put you on the planned loss rate in approximately 7 days.” Daily measurements allow new recommendations or congratulations, etc. to be generated from each day's fresh calculations that after the third day, FIG. 8, include slope of the mean basal weights. With increasing data, monitor displays are automatically scaled to the times and weights as in FIG. 11. Normally an expended scale for a week, such as FIG. 8 is first shown at each basal weight for 1-minutes, followed by a display scaled to cover collected data as in FIG. 11, which also has a reset which will be described later. Initialization process 408, consist of repeating the first basal weight until 3 values of zero difference are obtained². This value becomes the starting value for both the programmed or desired change, typically a linear loss of 0.1 lb/day, and also the actual weight change. On this first day a distinctive dot on a suitably scaled graph will appear on a L.E.D. or other screen for 1-2 minute, FIG. 5. Also some brief operational instructions may be given by the monitor voice, e.g. “Your weight is X lb. Now drink a glass of water and see what happens”. After the basal weight is taken as many additional measurements as desired may be made during the day, Step D. These additional values will be displayed in digital format only, or in the digital format plus voice. An important part of the The present system program is such patient self-education on effects of various dietary intakes and of various activities. This educational process will be augmented by an instruction manual.

On initial usage, during the first day, within the weighing window, before eating and after voiding, the user weighs himself/herself at least three successive times, until 3 successive weights differ one from the other by ±0.1 lb. This weigh represents the initial point or origin of the prescribed rate of loss line, the target weight trend or Rx line, which is then displayed graphically as shown in FIG. 8. This initial weight is also stored for additional display, for recall or for transfer to other units such as the host computer. The initial weight and the user initialization process is also used to establish a user identification profile, if the monitor is designed to multiple users. If during the following day, this user weighs himself/herself again and is within the weight tolerance, typically ±3%, the weight will be measured and either displayed graphically if in the accepted time frame and stored or otherwise displayed digitally and discarded. Taking weight measurement within the same time frame every day and accepting a new weight only if it is within the preset weight value range, also aids in insuring that the patient is in the standard weight taking condition, newly awakened, voided and absent of any solid or liquid intake. Additional spot weights that fall within the specified tolerance may be taken and displayed with the patient's ID at any time. As described, initially weights are then taken for two additional days to establish a mean basal weight. Meaningfull weight control begins on the 4^{th} day when another mean of the previous three weighing is calculated. A line segment is then established between the two points and a slope determined and compared to the desired slope. At this point weight control feedback to the patient can begin on the visual display and/or from the speaker via a synthetic voice message. The tone and gender of the voice will have been determined and programmed into the program loaded into the domicile monitoring unit from the expert computer from the evaluation and questionnaire data obtained from the patient. This preset tone and gender will then be used to generate an appropriate message based on the weight data in an appropriately toned synthesized voice. For example, if the weight data shown no change or a positive slope, the message may suggest reducing the quantity of a given food (tailored specifically to the patient based on questionnaire answers) or an increase in a given physical activity (again tailored specifically to the patient based on questionnaire answers). Successive basal weights and corresponding sliding averages and slopes will give a more accurate picture of program compliance and will allow the monitoring unit to better construct appropriate messages designed to assist the patient in achieving the desired final weight.

Some of the key features of the Caloric-Balance approach of this invention include at least: (1) realistic long-term goals for a lifetime, not quick changes to life style; (2) no exercise or diet prescriptions are made, rather the patient is educated to modulate his existing lifestyle; and (3) the educated patient is then left with the tools necessary to monitor and modulate his/her weight. It is anticipated that 40-50% will respond to this program. For those who do not, there are additional paths.

Referring now to FIG. 9, a plot of a model target weight control trend, actual weight data and actual trend are shown. This plot is a black and white example of a color display that would be produced by the weight measuring and feedback apparatus. A typical two week epoch using an actual example is shown. Daily weights (closed circles ● and dashed line) are shown starting with an initial or standing weight taken on day 1 through a current weight taken on day 12. The current weight can be highlighted to distinct it from the other weight data by color, flashing, or any other graphical highlighting technique. The weight measuring and feedback apparatus generally will issue minimal recommends during daily weighings unless there are large departures from the trend. Thus, the apparatus would recommend modifications to intake and/or output based on the data points at days 8 and 9. In the case of day 8, the apparatus would recommend that the weight loss is too large and a modest increase in food intake is warranted, while in the case of day 9, the apparatus would recognize an over compensation of the recommendation of day 8 and recommend a modest correction to either intake or output. The desired trend, the weight control program goal trend, is always displayed (solid line). The first trend point, based on a three day average value, appears at day 3 and is the average of days 1-3. The second trend point is the average of days 2-4, etc. The apparatus will issues few recommendations provided no significant differences are detected between the daily weight values and the trend values and/or the target values. However, the apparatus can issue encouraging statements tailored to the patient, if the patient profile deems such encouragement valuable in assisting the patient to adhere to the program. As stated above, the apparatus warned the patient on day 8 against moving too quickly to reduce weight, which resulted in an overcorrected, but subsequently the patients actual trend and actual weight followed more closely the target trend line. This was not a particularly motivated subject, but illustrates how effective small incremental control can be.

Non-Compliant Patients

For patients who do not comply with the weight control programs of this invention as set forth above, the reason for non-compliance must be sought. Two common reasons are denial and food problems or eating disorders. The patients who contend that they “run hard for 3 miles all the time” and then work hard or the patients who content that they “don't really eat anything” are all too familiar. A percentage of these patients may be made to face reality by objective monitoring. Physical activity can be monitored to almost any degree needed for days or weeks by means of a few coin sized sensors attached to the body equipped with wireless communication hardware and software as is well known in the art. The communication can be to a small recording unit carried by the patient (cell phone size) or to a remote location. The recorded data can then be reviewed by physician or expert and patient, once or on a set schedule, for an objective assessment of exercise duration and type so that output compliance may be monitored. A similar approach can be used to monitor ingestion of food and drink and a similar evaluation done. Such an active intervention programs increase overall success by about 5% to about 25%.

Other problems are in the food area. Many people are simply “hooked” on existing fast foods, a relatively low cost source of food. If foods that compete in taste and portability can be developed, this might salvage another incremental percentage. For example an array, not a diet, of such foods, that do not require refrigeration and that can be taken to work or eaten more formally at home might compete with fast food. This would also meet the requirements of working mothers, etc. who most often rely on fast or take-out foods. Again, such foods are not diet foods, but simply competition to undesirable foods. Such foods would also make control of caloric balance simpler for the group that are “hooked” on fast food.

Finally, there is no way that any program can salvage all overweight individuals from the combination of psychological problems, self-indulgent products supported by billions of dollars in advertising, and attractive low cost foods in such abundance.

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

1. A system for implementing an individual weight control program comprising:
an individualized weight control program including patient data, patient target weight, and a patient target weight trend line terminating in at the target weight;
a home monitoring unit including:
a digital processing unit (DPU) having:
a digital processor;
memory;
a display;
an audio output component;
an operating system;
communication hardware and software;
an A/D converter;
a portable mass storage device;
optional mass storage device;
optional audio input component;
a scale having a ±0.1 lb resolution in electrical communication with the DPU, and
a power supply for supply electric power to the DPU and the scale,
where the monitoring unit is designed to assist in patient compliance with the program and where the individualized weight control program is loaded into the memory of the DPU and is designed to monitoring of a patient's compliance on a daily basis.

2. The system of claim 1, wherein the communication hardware and software includes wired or wireless internet communication routines.

3. The system of claim 1, wherein the monitoring unit is designed to receive daily weight data within a weighing time window, where the weight data is of a patient in the nude, after voiding and before ingestion of liquids and/or solids, to compute a three day mean average weight and to display the weight data, three day mean average weight data, a three day mean average weight trend line, and the target weight control trend line on the display.

4. A method for implementing an individual weight control program comprising the steps of:
consulting with a physician or expert or other healthcare provider to develop an individualized weight control program;
gathering patient specific data,
entering the patent specific data into a host computer,
determining a weight goal and entering the weight goal into the host computer;
reviewing the data and entering in additional relevant patient specific data;
generating the individualized weight control program using software in the host computer based on the weight goal and patient data including eating and exercise habits,
reviewing the individualized weight control program,
if needed, modifying the patient data and generating a modified weight control program until an acceptable weight control program is approved,
transferring the individualized weight control program to a monitoring apparatus including:
a digital processing unit (DPU) having:
a digital processor;
memory;
a display;
an audio output component;
an operating system;
communication hardware and software;
an A/D converter;
a portable mass storage device;
optional mass storage device;
optional audio input component;
a scale having a ±0.1 lb resolution in electrical communication with the DPU, and
a power supply for supply electric power to the DPU and the scale, initializing the monitoring apparatus;
taking daily weighings within a daily weighing window, in the nude, after voiding and before ingestion of liquids and/or solids;
computing a three day weight average,
displaying a target weight trend line, the daily weighings, the three day weight averages, and a three day weight average trend line on the display;
comparing, on a daily basis, the trend line weight to the three day weight average value on a daily basis and generating audio messages designed to assist the patient in complying with the weight control program;
periodically, transferring the weight data to the host computer, and
periodically reviewing the weight data with the patient's physician or expert or other healthcare provider.

5. The method of claim 4, further comprising:
modifying the weight control data based on the weight data.

6. The method of claim 4, further comprising:
setting a slop of the weight control program trend line to zero, when the target weight is achieved.

7. A method for designing, monitoring and assisting a weight control program including the steps of:
entering patient identification data and a weight goal into a monitoring unit comprising:
a digital processing unit (DPU) having:
a digital processor;
memory;
a display;
an audio output component;
an operating system;
communication hardware and software;
an A/D converter;
a portable mass storage device;
optional mass storage device;
optional audio input component;
a scale having a ±0.1 lb resolution in electrical communication with the DPU, and
a power supply for supply electric power to the DPU and the scale;
constructing an individualized weight control program;
initializing the monitoring apparatus;
taking daily weighings within a daily weighing window, in the nude, after voiding and before ingestion of liquids and/or solids;
after three daily weighings, computing a three day weight average;
displaying a target weight trend line, the daily weighings, the three day weight averages, and a three day weight average trend line on the display; and
comparing, on a daily basis, the trend line weight to the three day weight average value on a daily basis and generating audio messages designed to assist the patient in complying with the weight control program.

8. The method of claim 7, further comprising the steps of:
prior to the entering step, consulting with a non-physician healthcare provider;
gathering patient physiological data and entering the physiological data into a host computer;
generating a patient profile from the entered physiological data; and
reviewing the patient profile and entered physiological data.

9. The method of claim 8, further comprising the steps of:
modifying the patient profile and entered physiological data.

10. The method of claim 8, further comprising the steps of:
after the comparing step, periodically, transferring the weight data to the host computer, and
periodically reviewing the weight data with the patient's physician or expert or other healthcare provider.

11. The method of claim 7, further comprising the steps of:
prior to the entering step, consulting with a physician;
gathering patient physiological and psychological data;
performing a physical examination;
entering the patent physiological and psychological data and the physical examination data into a host computer,
generating a patient profile from the entered physiological and psychological data and the physical examination data, and
reviewing the patient profile and entered physiological and psychological data and the physical examination data.

12. The method of claim 11, further comprising the steps of:
modifying the patient profile and entered physiological and psychological data and the physical examination data.

13. The method of claim 11, further comprising the steps of:
after the comparing step, periodically, transferring the weight data to the host computer, and
periodically reviewing the weight data with the patient's physician or expert or other healthcare provider.
